(19) United States
(12) Patent Application Publication
(10) Pub. No.: US 2003/0100944 A1
(43) Pub. Date: May 29, 2003

(54) Vascular graft having a chemicaly bonded electrospun fibrous layer and method for making same

(76) Inventors: Olga Laksin, Scotch Plains, NJ (US); George W. Du, Sparta, NJ (US)

Correspondence Address: Datascope Corp., 14 Philips Parkway, Montvale, NJ, 07645, US

(21) Appl. No.: 09/996,103

(22) Filed: Nov. 28, 2001

(51) Int. Cl. A61F 2/06

(52) U.S. Cl. 623/1.44

(57) ABSTRACT

A vascular graft comprising a traditional graft material and an electrospun fibrous layer. The solvent used to reduce the material for the electrospun layer is also capable of reducing the graft material to a liquid solution. The electrospun layer is chemically bonded to the graft material, without adhesives, by either spraying the graft with the solvent prior to electrospinning or by assuring that a sufficient amount of residual solvent reaches the graft while electrospinning.

### BACKGROUND OF THE INVENTION

1. Field of the Invention

The invention generally relates to implantable prostheses and the like and to methods for making same. More particularly, the invention relates to a vascular graft consisting of a woven or knitted fiber or other traditional graft material having an integrated inner and/or outer thin layer of much finer fibers and a method for making same.

2. Description of the Prior Art

Various synthetic vascular grafts have been proposed to replace, bypass, or reinforce diseased or damaged sections of a vein or artery. Commonly, the grafts have been formed from knitted or woven continuous filament polyester fibers, such as Dacron fibers (Dacron is a registered trademark of Dupont Inc.), and from expanded polytetrafluoroethylene (PTFE).

The performance of vascular grafts is influenced by a variety of characteristics such as strength, permeability, tissue ingrowth, and ease of handling. A graft should be sufficiently strong: (a) to prevent the sidewalls from bursting when blood is flowing through the device even at high blood pressures; and (b) to maintain the patency of the vessel lumen. Furthermore, the graft material must be sufficiently impervious to blood to prevent hemorrhaging as blood flows through the graft.

Expanded grafts are inherently leak resistant. Woven and knitted grafts, on the other hand, may require sealing of the openings between adjacent interlacings to prevent blood leakage. Sealing of said openings may be accomplished through a pre-clotting procedure. Pre-clotting involves immersing a woven or knitted graft in the patient's blood and then allowing the graft to dry until the interstices in the graft fabric become filled with the clotted blood. Another common technique for sealing the above mentioned openings is to coat the graft with an impervious material such as albumin, collagen, or gelatin. Tissue ingrowth through the interstices of the graft is believed to nourish and organize a thin neointima lining on the inner surface of the graft, preventing clotting of blood within the lumen of the graft, which could occlude the graft. A velour surface may be provided on the outer surface of a woven or a knitted graft to encourage tissue infiltration. The pore size of a graft also influences tissue ingrowth. Although larger openings facilitate tissue penetration, pre-clotting or coating of the graft may be adversely affected as pore size increases.

Ease of handling is another important feature of a vascular graft. A flexible and conformable graft facilitates placement of the prosthesis by the surgeon. The diameter of Dacron fiber is generally in the order of 10-20 microns. To survive the severe textile processing, each yarn bundle must consist of a large number of fibers, i.e. larger than 20 fibers. Increased elasticity, particularly of woven grafts has been achieved by crimping the graft. Crimping also improves resistance to kinking when the graft is bent or twisted. Woven and knitted grafts generally have been formed from continuous filament polyester yams, which typically are textured prior to fabrication to impart bulk and stretch to the vascular graft fabric. A technique known as false twist texturizing has been employed which involves the steps of twisting, heat setting, and then untwisting the continuous multifilament yams, providing substantially parallel, wavy filaments.

Graft selection for a particular application has therefore involved trade-offs and compromises between one or more of the above properties. Expanded PTFE grafts provide strong structures which are non-porous and impervious to blood leakage. The absence of pores, however, precludes tissue ingrowth. Expanded PTFE grafts also may be stiff and nonconforming which detrimentally affects handleablity. Knitted grafts have attractive tissue ingrowth and handleability features. The porous structure of knitted grafts, however, requires that the graft be pre-clotted or coated to prevent hemorrhaging. Woven grafts are less porous than knitted grafts and may not require pre-clotting or coating. The tightly compacted weave structure, however, may provide a stiff prosthetic which is not as conformable or as easily handled as is a knitted graft.

In light of the above, attempts have been made to make electrospun vascular grafts, see for example An Elastomeric Vascular Prosthesis, D. Annis et al, Vol. XXIV Trans. Am. Soc. Artif. Intern. Organs, 1978, pages 209-215. The reduced fiber size produced by electrospinning yield many desirable graft properties including low blood permeability, high porosity which facilitates tissue ingrowth and biological healing, and enhanced interaction between the outer surface of the graft and surrounding tissue. Unfortunately, due to the very small size of the fibers (usually less than 1 micron) conventional textile methods of processing are not useful and devices made entirely out of non-oriented or partially oriented fibers lack sufficient burst strength and mechanical sturdiness.

U.S. Pat. No. 5,116,360, issued to Pinchuk et al., discloses an additional support layer/component to compensate for the above-described deficiency. Using a graft having a conventional material in combination with an electrospun layer ensures sufficient mechanical strength while still providing for some of the benefits of an electrospun graft. However, use of the graft in combination with an electrospun layer introduces the problem of bonding the graft to the electrospun layer. Pinchuk et al. bond the graft to the electrospun layer using an intermediate layer having a melting temperature lower than both the graft and the electrospun layer. Upon raising the temperature above the melting point of the intermediary layer but below that of the graft and the electrospun layer, the intermediary layer melts and bonds the graft to the electrospun layer.

U.S. Pat. No. 6,165,212, issued to Dereume et al., describes encapsulation and anchoring of one graft layer between two nanofibrous deposits by means of heat-welding or adhesives, such as hot-melts, primers, and chemical adhesives.

All of the prior art bonding methods suffer from a major disadvantage. Namely, they all require the use of adhesives or primers. Excess adhesives or primers must be removed after bonding, thus adding an additional step to the manufacturing process. Furthermore, the very presence of the adhesive or primer in the body may constitute additional risk of some toxic reaction and may also block the pore channels within the graft, causing deterred healing.

While the known graft to electrospun layer bonding methods may be suitable for the particular purpose employed, or for general use, they would not be as suitable for the purposes of the present invention as disclosed hereafter.

### SUMMARY OF THE INVENTION

Accordingly, it is an object of the invention to produce a graft having a firmly connected electrospun fibrous layer.

The present invention comprises a synthetic fibrous vascular graft with improved surface morphology and a method for manufacturing said vascular graft. More particularly, the invention relates to a graft having a thin layer of much finer, preferably sub-micron size, fibers of the same or different material, on the outer and/or inner surface of the vascular graft in order to promote optimal tissue response. The solvent used to reduce the material used for the electrospun layer is also capable of reducing the graft material to a liquid solution. The electrospun layer is chemically bonded to the graft material by either spraying the graft with the solvent prior to electrospinning or by assuring that a sufficient amount of residual solvent reaches the graft while electrospinning.

Note that other than vascular grafts the bonding method of the present invention can be use to bond a fibrous electrospun layer to any article that can be made a depository of electrospun fibrous material, i.e. any substrate, including but not limited to articles of clothing, blood filters, heart valves, artificial tissue scaffolds, heart pumps or any other prosthetic devices for implantation into the body, so long as the material is reducible to a solution form in the chosen solvent.

### BRIEF DESCRIPTION OF THE DRAWINGS

In the drawings, like elements are depicted by like reference numerals. The drawings are briefly described as follows.

FIG. 1 is a scanning electron microscope (SEM) microphotograph taken of an electrospun fibrous graft layer spun as per example 1 with ×575 magnification.

FIG. 1A is a SEM microphotograph taken of an electrospun fibrous graft layer spun as per example 1 with ×5450 magnification.

FIG. 2 is a SEM microphotograph taken of an electrospun fibrous graft layer spun as per example 2 with ×585 magnification.

FIG. 2A is a SEM microphotograph taken of an electrospun fibrous graft layer spun as per example 2 with ×6050 magnification.

FIG. 3 is a SEM microphotograph taken of a transverse cross section of a graft having a graft layer and an electrospun fibrous layer with ×2000 magnification.

### DETAILED DESCRIPTION OF THE PREFERRED EMBODIMENTS

The present invention involves an electrospun fibrous layer chemically bound to a substrate, such as a vascular graft. Accordingly, for clarity purpose the following detailed description is broken down to five sections which describe in detail the basic electrospinning process, the electrospun materials used to form the fibrous layer, the graft construction and material, the bond between the graft and the electrospun fibrous layer, and the properties of the fibrous layer.

Basic Electrospinning Process

The basic process of electrospinning is described in detail in U.S. Pat. No. 4,323,525, issued to Bornat, herein incorporated by reference in its entirety. The process involves the introduction of a polymer solution into an electric field whereby the liquid is caused to produce fibers. After being drawn from the liquid the fibers harden and may be collected upon a suitably charged surface.

In general, the apparatus needed to carry out the electrospinning of the present invention, and thereby produce the micro or nanofibers used to make the graft of the present invention, includes a delivery point, a delivery means, an electric field, and a capture point.

The delivery point is simply a place where at least one droplet of the fiber or spinning solution can be introduced or exposed to an electric field.

The capture point is simply a place where the stream or jet of polymeric liquid, or more generally spinning solution, can be collected. It is preferred that the delivery point and the capture point be conductive so as to be useful in creating the electric field. It should be understood, however, that the invention is not limited to this type of configuration or setup inasmuch as the delivery point and capture point can be non-conductive points that are simply placed within or adjacent to an electric field. It is preferred, however, that the electrospinning apparatus is configured so that the spinning solution is pulled horizontally through space.

As for the electric field, the person skilled in the art should appreciate that the electrostatic potential should be strong enough to overcome gravitational forces on the spinning solution, overcome tension forces of the spinning solution, provide enough force to form a stream or jet of solution in space, and accelerate that stream or jet across the electric field. As the person skilled in the art will recognize, surface tension is a function of many variables, including but not limited to the type of polymer, the solution concentration, and the temperature.

Any convenient delivery means may be employed to bring the spinning solution into the electrostatic field. For example, the spinning solution may be fed into the electrostatic field through a nozzle, or through a syringe needle, or a spinneret. It will be appreciated that multiple nozzles, syringes, spinnerets or other delivery means may be used to increase the rate of fiber production. Similarly, the size of the orifice, i.e. the hole through which the spinning solution flows, can be varied to further control the rate of fiber production. The delivery means may also employ a slot or a perforated plate for spraying the solution through.

The flow of spinning solution can be controlled by a pump or can be adjusted through a combination of parameters such as but not limited to for example, solution viscosity, orifice size, and orifice position. Lower solution viscosities and smaller orifice sizes tend to produce smaller fibers. Control of the level of flow of spinning solution via the delivery means into the electrostatic field is important to achieve desirable fiber sizes and optimal concentration of residual solvent.

Another important aspect of electrospinning of spinning solution is the density of electrostatic charge in the solution. While in many applications it may be sufficient to apply the electrostatic potential directly to the delivery means, for example the nozzle, it is preferred to actually placing an electrode into the spinning solution, for example into the syringe reservoir, so as to increase the charge density of the spinning solution. One may use an electrode made from metal having the shape of a rod, thin plate, or a brush having metal fibers twisted together. Any type of the electrode is acceptable, however, in order to increase the charge density of the spinning solution it is preferred to maximize the charged surface area of the electrode.

The electrostatic potential employed to produce fibers via the electrospinning process is generally within the range 5 Kv to 1000 Kv, conveniently 10-100 Kv, and preferably 10-50 Kv over a distance of approximately 5-20 inches. Any appropriate method of producing the desired potential may be employed. It is also a matter of choice which charge to apply to the spinning solution and which to the delivery point.

Electrospun Materials Used to Form Thin Fibrous Layer

Materials suitable for use in the electrospinning process include virtually any polymer that can be reduced to a solution form. The fibers can be made from any of biodegradable or non-biodegradable polymers that are suitable for implantation into the body. Useful non-biodegradable polymers include, but are not limited to, polyesters, polyurethanes (polyether-, polyester-, silicone- and polycarbonate block co-polymers), polyolefines, and polyetheramides. Useful biodegradable polymers include, but are not limited to, polyglycolic acid (PGA), polylactic acid (PLA) and derivatives thereof, polycaprolactone, polyhydroxybutyrate, polyethyl glutamate, polydioxanone, poly (ortho esters), polyanhydride, polyamino acids and backbone-modified “pseudo”-poly (amino acids), as well as natural material derived products such as collagen, cellulose, and hylans. Note that any biocompatible polymer that can be reduced to a solution form can be used for this application.

Graft Construction and Material

Conventional artificial vascular grafts are generally tubular in shape and are generally made of porous, woven, knitted or braided materials, such as nylon, polyester, polytetrafluoro ethylene (PTFE), polypropylene, polyacrylonitrile, polyurethanes, etc. The long-term stability of a conventional artificial vascular graft having an electrospun fibrous layer is contingent upon its mechanical integrity, and therefore an excellent bonding between the layers becomes essential.

Note that the present invention extends beyond the field of vascular grafts as it can be used to bond a fibrous electrospun layer to any article that can be made a depository of electrospun fibrous material, i.e. any substrate, including but not limited to articles of clothing, absorption devices (such as gauze and tampons), filters (such as an air, protein, or blood filters), heart valves, artificial tissue scaffolds, heart pumps or any other prosthetic devices for implantation into the body, so long as the material is reducible to a solution form by the chosen solvent.

Bond Between Graft and Fibrous Layer

It has been discovered that choice of the solvent and use of the solvent on the graft can have a dramatic effect on the strength of the bond between the graft and the electrospun layer. Specifically, in order to chemically bond, and thus assure a strong connection between, the graft and the electrospun fibrous layer it is important (1) to chose a solvent capable of reducing to a solution not only the material for electrospinning but also the graft itself and (2) applying a solvent prior to electrospinning or to assure that the electrospinning process parameters are chosen to assure a sufficient amount of residual solvent.

Note that residual solvent is the remaining unevaporated solvent in the spinning solution upon initial contact with the graft. Note also that although it is possible to reduce the molecular weight of the polymer by immersing it into a given solvent, the term reduce as used in this application specifically refers to a change of state from solid to liquid. Note further that the term solvent as used herein may comprise a single solvent or multiple different solvents mixed together.

When spinning polyurethane onto a PET graft, such as a Dacron graft, for example, one should use a solvent capable of reducing both polyurethane and PET to a solution. This may seen to be an unnecessary requirement given the fact that the graft material is not being electrospun, rather it is being spun on to, and thus, does not have to be reducible to a solution in the solvent. However, the inventors of the present invention have discovered that using a solvent, capable of reducing the graft to a solution, and assuring that sufficient solvent reaches the graft, results in a change of the surface chemistry of the graft providing for an adhesive-free chemical bond between the graft and fibrous layers deposited on the graft, without affecting bulk properties of the graft.

Despite the general practice in the art of choosing the electrospinning parameters so as to assure that the solvent evaporates from the spinning solution before it reaches the graft (see for example, col. 7, lines 19-21 of U.S. Pat. No. 6,110,590, issued to Zarkoob et al.), given the discovered importance of the solvent in creating a strong bond between the graft and the spun fibrous layer, the spinning solution should contain sufficient residual solvent when finally reaching the graft or alternatively the graft should be coated or sprayed with the solvent prior to electrospinning.

One can vary and control the parameters of the electrospinning process to assure a sufficient level of residual solvent. Note that the amount of residual solvent necessary will depend on the given design situation.

On the one hand, the required amount of residual solvent depends on the design end properties of graft, e.g. permeability and porosity, and the type of tissue response one desires to enhance and promote. One can design for a surface modification layer, for example to work as a conduit for endothelial tissue growth, or for additional construction layer. In the case of a highly permeable graft one may want to apply a thicker constructional layer to lower permeability. Furthermore, if there is a desire to encourage transmural growth one needs to design for larger pores which in turn require the use of a constructional layer of larger fibers.

On the other hand, the required amount of residual solvent will be defined by the choice of the materials and solvent. The more aggressive the solvent the less solvent necessary for a given application. Furthermore, the configuration of the graft, e.g. woven, knitted, dense or loose substrate, and the degree of susceptibility of the graft material to the given solvent also plays a role in the required amount of residual solvent.

With respect to the delivery means, one can use any delivery device, including a nozzle, have an opening through which the spinning solution will be drawn. The larger this opening the higher the rate of fluid drawn into the electric field and the higher the level of residual solvent. With regard to the electric field, the higher the intensity the lower the level of residual solvent. With regard to the opening size, the larger the opening size the higher the concentration of residual solvent and the larger the size of fibers. At the same time, however, use of a smaller opening may by default also increase residual solvent concentration. This is so because one needs to use of a lower concentration of polymer in the spinning solution when using a smaller opening. This is so because of the requirement of uniform introduction of spinning solution into the electric field which in turn requires the use of a lower concentration of polymer in the spinning solution when using a smaller opening. With regard to the distance between the delivery point and the capture point, increased distances tend to reduce residual solvent concentration without affecting fiber size. With respect to the spinning solution, the higher the solvent to fiber ratio the higher the level of residual solvent. Furthermore, addition of a third material, such as but not limited to Trifluoroethanol (TFE), may delay the evaporation of the solvent and thus increased the amount of residual solvent, see example three below. The third material may include, but is not limited to, an additional solvent, a viscosity adjuster, or substances that increase the electrostatic charge density of the spinning solution. Thus, control parameters of the electrospinning process should be considered to assure sufficient residual solvent.

As indicated above, one can control the parameters of the electrospinning process to assure a predetermined level of residual solvent throughout the electrospinning process. Alternatively, one can start the electrospinning process with an elevated solvent to fiber ratio, to assure a strong bond between the fibrous material and the graft, and then taper down to a lower level. In still yet another embodiment, one can apply to the graft, via electrospraying or another method known in the art, pure solvent prior to electrospinning the spinning solution. Application of pure solvent may be more desirable when spinning larger fibers. This is so because there is a reduced initial concentration of solvent in the spinning solution containing large fibers.

The concentration of the fiberizible polymer should be controlled to provide for adequate fiber structural properties. Furthermore, to allow for efficient spinning the spinning solution should have an appropriate viscosity and speed of fiber hardening.

The amount of residual solvent necessary to create the desired chemical bond between the graft and the electrospun layer will vary depending on the given design situation, as indicated above. In all situations, however, the amount of residual solvent and the initial application of the extra solvent should be controlled so as to assure a sufficient level for bonding but not so much as to cause damage to the graft and/or completely dissolve the spun fibers on the graft. Furthermore, the amount of residual solvent should be controlled so that the resultant graft maintains its desired permeability and morphology.

Three illustrative examples of electrospinning parameters are provided. Note that in all cases the graft material is reducible to a solution in the chosen solvent. Furthermore, the parameters are chosen in examples one through three to assure sufficient residual solvent and in example three to provide for an initial spraying of solvent. Specifically, in example one the needle and solvent concentration were specifically chosen to control the rate of solvent evaporation, i.e. amount of residual solvent. The smaller the needle the faster the solvent evaporates and the less residual solvent created. In example two an effort was made to produce a fibrous layer having larger fibers in the range of approximately 500 nm to 3 microns. Accordingly, Trifluoroethynol was added to the spinning solution of example one to control the evaporation of the spinning solution. In example three, prior to electrospinning the spinning solution pure solvent was sprayed onto the graft.

### EXAMPLE ONE

The delivery means used comprised a pair of 20-cc glass syringes both containing a PET spinning solution. One syringe had a 15-gauge metal needle and the other had a 21-gauge metal needle. Note that the larger the needle gauge the smaller the diameter of the corresponding needle opening. The 15-gauge needle was used to assure a higher level of residual solvent required for bonding purpose, and the 21-gauge needle was used to produce smaller fibers required for the electrospun fibrous layer of the graft. The capture point comprised a Dacron vascular graft mounted on a rotating mandrel. The syringes were place on either side of the graft. The distance between a tip of each needle and the graft was approximately 10 inches. A positive potential was applied to the spinning solutions though immersed electrode metal brushes connected to a power source. The counter electrodes were grounded together with the mandrel. The spinning solution comprised 10% wt. PET in HFIP (Hexafluoro-Isopropanol). The potential difference between the solutions and the spinning mandrel was 25 kv. The spinning solution was electrospun for approximately four minutes.

### EXAMPLE TWO

All the parameters were kept same as in example one except two 18-gauge metal needles were used and Trifluoroethanol was added to spinning solution of example one to control the evaporation of the spinning solution. The weight ratio of the spinning solution of example one and Trifluoroethynol was nine to one. Compared with the 21-gauge needle used in example one, the 18-gauge needles produced larger fibers. Compared with the 15-gauge needle in example one, use of the 18-gauge needles resulted in a lower level of residual solvent. Trifluoroethanol (1 to 9 of HFIP) was added to slow down the evaporation of the spinning solution (TFE has a boiling point of 77-80 degree Celsius, HFIP has a boiling point of 59 degree Celsius). Adding TFE raised the level of residual solvent, and thus improved bonding of electrospun fibers to the graft.

### EXAMPLE THREE

A 21-gauge metal needle was used to deliver pure solvent HFIP (Hexafluoro-Isopropanol) onto a surface of a Dacron graft for approximately one minute. After which a switch was made to a pair of syringes filled with the spinning solution, as detailed in example one. The spinning solution of example one was electrospun for approximately four minutes.

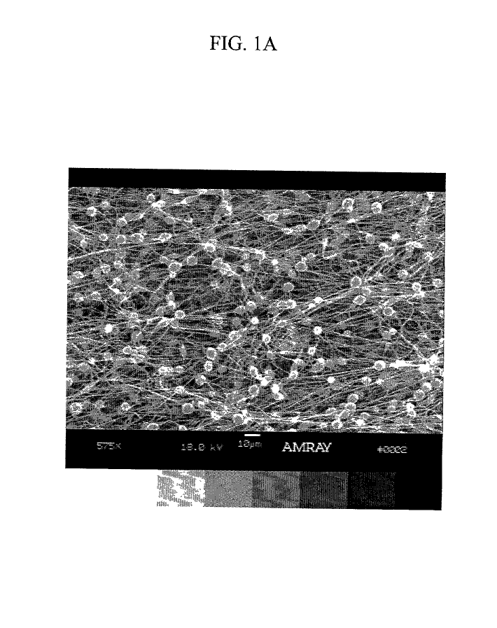

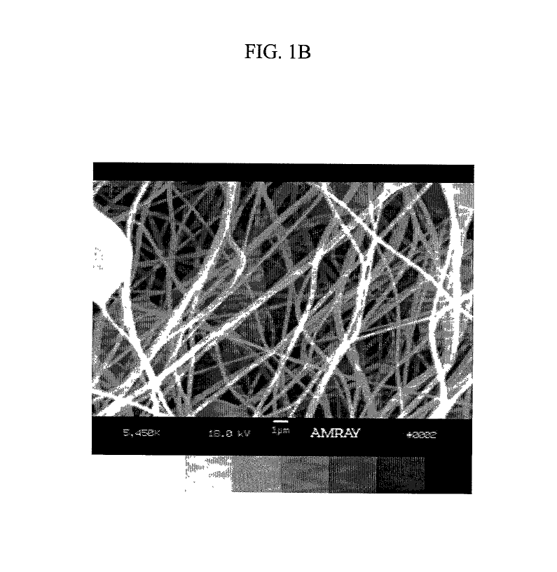

FIG. 1A is a SEM microphotograph, magnification ×575, of the electrospun fibrous layer created in example one. The fibrous layer has an average fiber size of approximately 500 nm with interfiber spaces of approximately 1-5 microns. Note that polymer droplets, in the range of approximately 5-10 microns, formed due to the excess of residual solvent, provide fiber-to-fiber bonding and fusion. Note also the random orientation of the fiber bundles and intertanglement of the individual fibers.

FIG. 1B is a higher magnification (×5450) SEM microphotograph of the same electrospun fibrous layer as shown in FIG. 1A. Note that individual fibers are not uni-directionally oriented, and thus have a high degree of three-dimensional intertanglement and inter-connectivity.

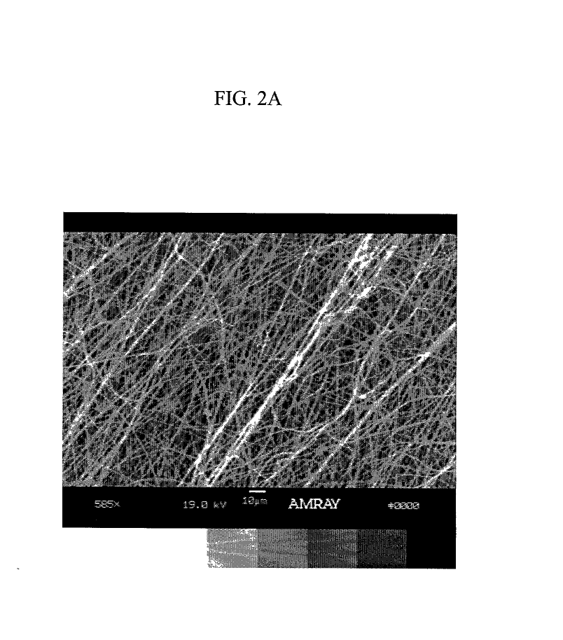

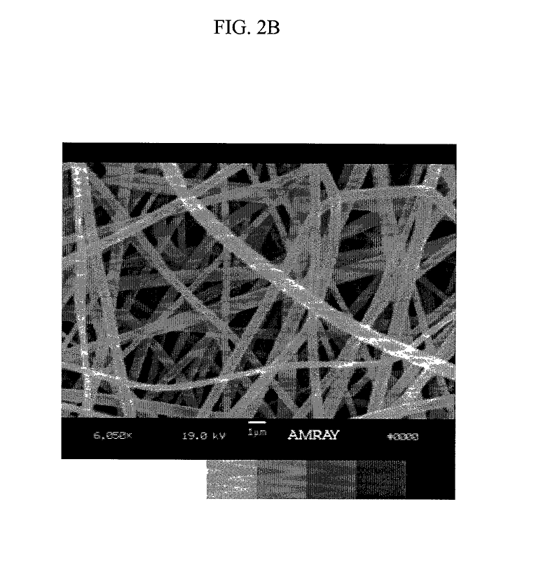

FIG. 2A is a SEM microphotograph, magnification ×585, of the electrospun fibrous layer created in example 2. The fibrous layer has an average fiber size of approximately 1 micron with interfiber spaces varying between approximately 5-10 microns. Note that the fiber bundles are predominantly oriented in one direction.

FIG. 2B is a higher magnification (×6050) SEM microphotograph of the same electrospun fibrous layer as shown in FIG. 2A. Note that individual fibers are not uni-directionally oriented and form a randomly intertangled network.

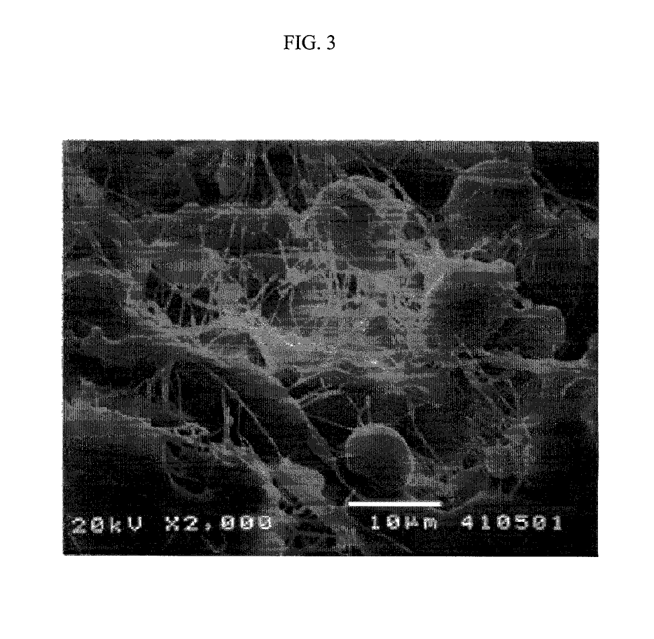

FIG. 3 is a SEM microphotograph, ×2000 magnification, taken of a transverse cross section of the electrospun graft created in example 1, focusing on the region where the graft material and the electrospun fibers are chemically bonded. The larger circular bodies are the graft material fibers, which range between 10 and 15 microns. The web-like stringy strands are the electrospun fibers. Note that the electrospun fibers terminate directly in the graft fibers, which indicates that the electrospun fibers and the graft fibers have chemically blended, thus forming an adhesive-free chemical bond.

Properties of Fibrous Layer

The fibrous layer on the graft of the present invention contains intertangled fibers, can be on the inner and/or outer surface of the graft, and can be formed having a gradient structure (multiple sublayers) along its thickness or length. For example, the blood contacting surface of the fibrous layer on the inside of the graft can be engineered to provide outstanding thrombogenicity, whereas the outside surface of the fibrous layer on the outside of the graft can be made of stronger material to provide strength. Note that the inside of a tubular graft may be coated by turning the tubular graft inside out and then electrospinning onto the inner surface. The thickness of each sublayer, the fiber diameter, pore size and distribution may vary from sublayer to sublayer in accordance with their targeted functions. Furthermore, the wall thickness of the fibrous layer may vary along the length of the graft.

As many apparently widely different embodiments of the present invention can be made without departing from the spirit and scope thereof, it is to be understood that the invention is not limited to the specific embodiments thereof except as defined in the appended claims.

What is claimed is:

1. A substrate comprising a substrate layer and one or more electrospun fibrous layers comprising at least some intertangled fibers, at least one of said fibrous layers being bonded to said substrate layer via an adhesive-free chemical bond.

2. A substrate comprising a substrate layer and one or more electrospun fibrous layers comprising at least some intertangled fibers, at least one of said fibrous layers being bonded to said substrate layer via an adhesive-free chemical bond, said chemical bond being formed as a result of exposing both the substrate and fibers in contact with the substrate to a solvent capable of reducing both the substrate and the fibers to a solution.

3. The substrate as claimed in claims 1 or 2 wherein the fibrous layer comprises at least two fibrous sublayers and wherein at least two of said fibrous sublayers have different properties resulting from the variation of at least one variable between sublayers, said at least one variable in the sublayer including thickness of each sublayer, fiber diameter, pore size, and pore distribution.

4. The substrate as claimed in claims 1 or 2 wherein the substrate layer is made from one or more materials selected from the group consisting of nylon, polyester, polytetrafluoro ethylene (PTFE), polypropylene, polyacrylonitrile, and polyurethane.

5. The substrate as claimed in claims 1 or 2 comprising an implantable medical device and wherein the substrate layer being an implantable medical device layer.

6. The substrate as claimed in claims 1 or 2 comprising a vascular graft and wherein the substrate layer being a vascular graft layer.

7. The substrate as claimed in claims 1 or 2 comprising a tissue scaffolding and wherein the substrate layer being a tissue scaffolding layer.

8. The substrate as claimed in claims 1 or 2 comprising a filter and wherein the substrate layer being a filter layer.

9. The substrate as claimed in claims 1 or 2 comprising an absorption device and wherein the substrate layer being an absorption device layer.

10. A method for forming a substrate comprising a substrate layer and an electrospun fibrous layer containing at least some intertangled fibers, said method comprising the step of electrospinning a spinning solution onto the substrate layer, said spinning solution containing a solvent and a polymer reduced to a solution in said solvent, said substrate layer being reducable to a solution by said solvent.

11. The method as claimed in claim 10 wherein the electrospinning is performed so as to assure a level of residual solvent sufficient to create a chemical bond between the substrate layer and fibrous layer.

12. The method as claimed in claim 10 comprising the step of applying a solvent to the substrate layer prior to electrospinning the spinning solution onto the substrate layer.

13. A method for forming a substrate comprising a substrate layer and a fibrous layer containing at least some intertangled fibers, comprising the steps of: (a) positioning a delivery means a predetermined distance away from the substrate layer, said delivery means containing a spinning solution, said spinning solution containing a solvent and a polymer reduced to a solution in said solvent, said substrate layer being reducable to a solution by said solvent; and (b) creating an electric field between the substrate layer and the spinning solution such that at least a portion of the spinning solution passes from the delivery means through the electric field towards the substrate layer.

14. The method as claimed in claim 13 wherein the electric potential is created by passing an electrode into the spinning solution.

15. The method as claimed in claims 10 or 13 wherein the substrate comprises an implantable medical device and the substrate layer comprises an implantable medical device layer.

16. The method as claimed in claim 10or 13 wherein the substrate comprises a vascular graft and the substrate layer comprises a vascular graft layer.

17. The method as claimed in claims 10 or 13 wherein the substrate comprises a tissue scaffolding and the substrate layer comprises a tissue scaffolding layer.

18. The method as claimed in claims 10 or 13 wherein the substrate comprises a filter and the substrate layer comprises a filter layer.

19. The method as claimed in claims 10 or 13 wherein the substrate comprises an absorption device and the substrate layer comprises an absorption layer.
